(19) United States
(12) Patent Application Publication
(10) Pub. No.: US 2016/0008336 A1
(43) Pub. Date: Jan. 14, 2016

(54) HDAC INHIBITORS FOR THE TREATMENT OF TRAUMATIC BRAIN INJURY

(71) Applicant: John S. KOVACH, (US)

(72) Inventor: John S. KOVACH, East Setauket, NY (US)

(73) Assignee: Lixte Biotechnology, Inc., East Setauket, NY (US)

(21) Appl. No.: 14/772,317

(22) PCT Filed: Feb. 27, 2014
(86) PCT No.: PCT/US14/18999

§ 371 Date: Sep. 2, 2015

Related U.S. Application Data
(60) Provisional application No. 61/772,966, filed on Mar. 5, 2013.

(51) Int. Cl. A61K 31/4406; A61K 31/166; A61K 31/167

(52) U.S. Cl. CPC A61K 31/4406; A61K 31/167; A61K 31/166

(57) ABSTRACT

The present invention provides a method of treating a subject suffering from traumatic brain injury comprising administering to the subject an effective amount of an HDAC inhibitor, structurally represented, thereby treating the subject suffering from traumatic brain injury.

Throughout this application various publications are referenced. The disclosures of these documents in their entireties are hereby incorporated by reference into this application in order to more fully describe the state of the art to which this invention pertains.

### BACKGROUND OF THE INVENTION

Traumatic brain injury (TBI), a form of acquired brain injury (clinically defined as neurological dysfunction following head trauma), has recently received increased attention within the media and medical literature. There are approximately 1.7 million documented TBIs in the United States each year, with current estimates being around 4 million cases per year (Faul, M. et al. 2010). Furthermore, TBI costs the U.S. more than $56 billion a year (Faul, M. et al. 2010). More than 5 million Americans, as a direct result of a TBI, require permanent assistance in performing activities of daily living (Faul, M. et al. 2010). TBI initiates a complex series of neurochemical and signaling changes that lead to brain tissue pathogenesis, including neuronal hyperactivity and dysfunction, excessive glutamate release, inflammation, increased blood-brain barrier (BBB) permeability and cerebral edema, and altered gene expression. Unfortunately, there is a paucity of accepted biomarkers for the diagnosis and/or prognosis of this disease and of proven pharmacological therapies for treatment. Moreover, human clinical trials aimed at testing potential neuroprotective therapies have not been successful to date (Dash, P. K. et al. 2010; Narayan R. K. et al. 2002; Kumar, A. et al. 2012).

Histone deacetylase inhibitors (HDACi) are a class of therapeutic drugs designed to regulate proteostasis. HDACi modulate cellular function by posttranslational modification of histones, transcriptional factors, and protein chaperones, including heat shock proteins (Hsp). More specifically, HDACis regulate protein expression by directly modulating gene transcription, while also altering protein degradation by changing the sensitivity of the unfolded protein response and decreasing the ubiquitination and proteasomal degradation of misfolded proteins. Though the exact mechanism behind these observed effects remains unknown, there has been an effort to develop histone deacetylase inhibitors (HDACi) as a viable anti-cancer treatment (Monneret, C. et al. 2007; Richon, V. M. et al. 2002; Gibson, C. L. et al. 2010). Suberoylanilide hydroxamic acid (SAHA or Vorinostat) use began over three decades ago. It causes growth arrest and death of a broad variety of transformed cells, both in vitro and in vivo, and at concentrations that have little or no toxic effects on normal cells. SAHA inhibits the activity of HDACs, including all 11 known human class I and class II HDACs, and was approved for sale in the U.S. in October 2006 for the treatment of cutaneous T-cell lymphoma (CTCL).

### SUMMARY OF THE INVENTION

The present invention provides a method of treating a subject suffering from traumatic brain injury comprising administering to the subject an effective amount of an HDAC inhibitor having the structure:

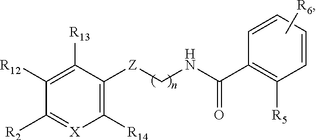

wherein
n is 1-10;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
Z is

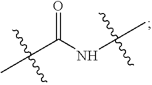 [SMILES: *NC(*)=O]

R₂ is H or NR₃R₄, wherein R₃ and R₄ are each independently H, C₁-C₆ alkyl, or C₃-C₈ cycloalkyl;
R₅ is OH or SH;
R₆, R₁₂, R₁₃, and R₁₄ are each independently H, OH, SH, F, Cl, trifluoromethyl, methoxy, or CO—R₁₅, wherein R₁₅ is alkyl, alkenyl, alkynyl, C₃-C₈ cycloalkyl, or aryl, or
a salt of the compound,
   thereby treating the subject suffering from traumatic brain injury.

### BRIEF DESCRIPTION OF THE FIGURES

FIG. 1. FDG-PET Image for Rat Brains. Brighter regions of the image show a greater response to the injected FDG, which indicates normal glucose levels and healthy blood flow. HDACi treated rat brain showed improved post-TBI glucose uptake.

FIG. 2. Gross Appearance following TBI. After HDACi (205) treatment, gross appearance of the TBI damage area was improved.

FIG. 3. H & E staining. TBI models shows increased cellular destructions and brain tissue damage compared to sham. After HDACi (205) treatment, increased reactive gliosis (arrow) and more active brain cells observed, indicating HDACi induced a positive effect upon brain tissue injury repair.

FIG. 4. Behavioral Motor Performance Test. Post-injury administration of HDACi (205, 10 mg/kg) improves behavioral performance in post-traumatic brain injured rats. Rats had to cross bridge (one meter bar) to test their balance and motor skills.

FIG. 5. (a) HDACi (205) decreases Pro-caspase 3 expression. (b) HDACi (205) increases p-AKT expression.

FIG. 6. Immunofluorescence. GFAP (green or triangled regions), Nestin (red or squared regions), and DAPI (blue, various regions). HDACi (205) treatment increased Nestin and GFAP expression following TBI, indicating increased injury repair response.

FIG. 7. Controlled cortical impact location over the left frontal cortex.

FIG. 8. DNA microarray analysis may display different fluorescence intensities for TBI samples at different cDNA intercept locations. A) normal tissue, B) TBI tissue, and C) TBI+HDACi.

FIG. 9. Table displaying experiment schedule following TBI surgery.

### DETAILED DESCRIPTION OF THE INVENTION

The present invention provides a method of treating a subject suffering from traumatic brain injury comprising administering to the subject an effective amount of an HDAC inhibitor having the structure:

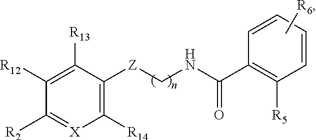

wherein
n is 1-10;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
Z is

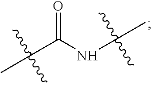 [SMILES: *NC(*)=O]

R₂ is H or NR₃R₄, wherein R₃ and R₄ are each independently H, C₁-C₆ alkyl, or C₃-C₈ cycloalkyl;
R₅ is OH or SH;
R₆, R₁₂, R₁₃, and R₁₄ are each independently H, OH, SH, F, Cl, trifluoromethyl, methoxy, or CO—R₁₅, wherein R₁₅ is alkyl, alkenyl, alkynyl, C₃-C₈ cycloalkyl, or aryl, or
a salt of the compound,
   thereby treating the subject suffering from traumatic brain injury.

In some embodiments, the method includes the compound:

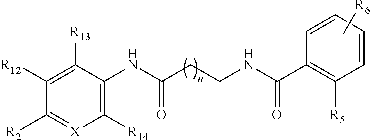

wherein
n is 1-9;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
R₂ is H or NR₃R₄, wherein R₃ and R₄ are each independently H, C₁-C₆ alkyl, or C₃-C₈ cycloalkyl;
R₅ is OH or SH; and
R₆, R₁₂, R₁₃, and R₁₄ are each independently H, OH, SH, F, Cl, SO₂R₁₅, NO₂, trifluoromethyl, methoxy, or CO—R₁₅, wherein R₁₅ is alkyl, alkenyl, alkynyl, C₃-C₈ cycloalkyl, or aryl.

In some embodiments, the method includes the compound:

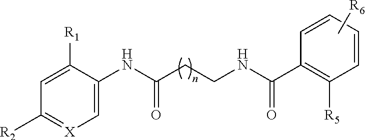

wherein
n is 1-8;
X is CH or N;
R₁ is H or OH;
R₂ is H or NR₃R₄, wherein R₃ and R₄ are each independently C₁-C₆ alkyl or C₃-C₈ cycloalkyl;
R₅ is OH or SH; and
R₆ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl.

In some embodiments, the method includes the compound:

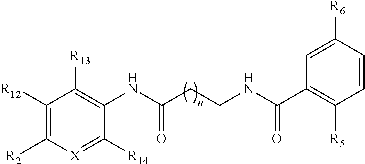

wherein
n is 1-9;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
R₂ is H or NR₃R₄, wherein R₃ and R₄ are each independently H, C₁-C₆ alkyl, or C₃-C₈ cycloalkyl;
R₅ is OH or SH; and
R₆, R₁₂, R₁₃, and R₁₄ are each independently H, OH, SH, F, Cl, trifluoromethyl, methoxy, or CO—R₁₅, wherein R₁₅ is alkyl, alkenyl, alkynyl, C₃-C₈ cycloalkyl, or aryl.

In some embodiments, the method includes the compound:

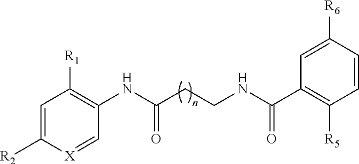

wherein
n is 1-8;
X is CH or N;
R₁ is H or OH;
R₂ is H or NR₃R₄, wherein R₃ and R₄ are each independently C₁-C₆ alkyl or C₃-C₈ cycloalkyl;
R₅ is OH or SH; and
R₆ is H, OH, SH, F, Cl, trifluoromethyl, methoxy, or CO—R₇, wherein R₇ is alkyl, alkenyl, alkynyl, or C₃-C₈ cycloalkyl, or aryl.

The method of claim 1, wherein the compound has the structure:

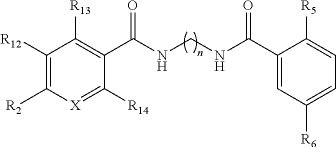

wherein
n is 1-8;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
R₂ is H or NR₃R₄, wherein R₃ and R₄ are each independently C₁-C₆ alkyl or C₃-C₈ cycloalkyl;
R₅ is OH or SH;
R₆, R₁₂, R₁₃, and R₁₄ are each independently H, OH, SH, F, Cl, trifluoromethyl, methoxy, or CO—R₁₅, wherein R₁₅ is alkyl, alkenyl, alkynyl, C₃-C₈ cycloalkyl, or aryl.

In some embodiments, the method includes the compound:

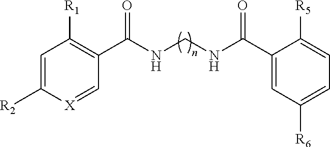

wherein
n is 3-8;
X is CH or N;
R₁ is H, OH or SH;
R₂ is H or NR₃R₄, wherein R₃ and R₄ are each independently C₁-C₆ alkyl or C₃-C₈ cycloalkyl; and
R₅ is OH or SH; and
R₆ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl, or
a salt of the compound.

In some embodiments, the compound wherein R₁ and R₂ are H, X is CH, R₅ is SH, R₆ is H, and n is 4.

In some embodiments, the compound wherein R₁ is OH, R₂ is H, X is CH, R₅ is OH, R₆ is H, and n is 6.

In some embodiments, the compound wherein R₁ is SH, R₂ is H, X is CH, R₅ is SH, R₆ is H, and n is 6.

In some embodiments, the compound wherein R₁ and R₂ are H, X is N, R₅ is SH, R₆ is H, and n is 4.

In some embodiments, the compound wherein R₁ is H, R₂ is NR₃R₄, wherein R₃ and R₄ are each C₁ alkyl, X is CH, R₅ is SH, R₆ is H, and n is 4.

In some embodiments, the compound wherein R₁ and R₂ are H, X is N, R₅ is SH, R₆ is Cl, and n is 4.

In some embodiments, the compound wherein R₁ and R₂ are H, X is N, R₅ is SH, R₆ is H, and n is 5.

In some embodiments, the compound wherein R₁ is H, R₂ is NR₃R₄, wherein R₃ and R₄ are each H, X is CH, R₅ is SH, R₆ is H, and n is 4.

In some embodiments, the compound wherein R₁ and R₂ are H, X is CH, R₅ is SH, R₆ is Cl, and n is 4.

In some embodiments, the compound wherein R₁ and R₂ are H, X is CH, R₅ is SH, R₆ is methoxy, and n is 4.

In some embodiments, the compound wherein R₁ and R₂ are H, X is CH, R₅ is SH, R₆ is H, and n is 5.

In some embodiments, the compound wherein R₁ and R₂ are H, X is CH, R₅ is SH, R₆ is H, and n is 6.

In some embodiments, the compound wherein R₁ and R₂ are H, X is CH, R₅ is SH, R₆ is H, and n is 9.

In some embodiments, the method includes the compound wherein the structure is:

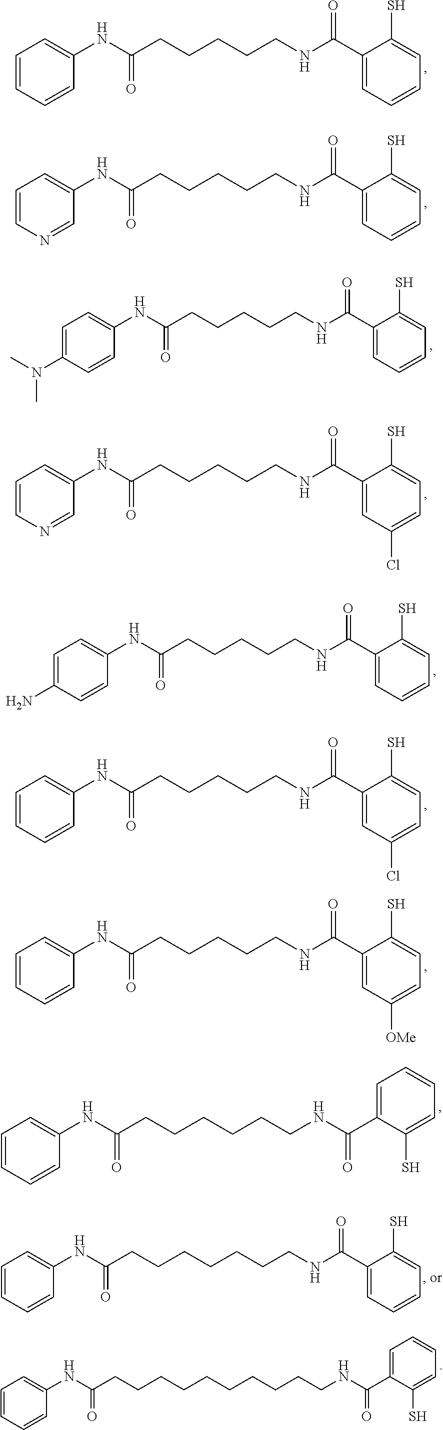

In some embodiments, the method includes the compound wherein the structure is:

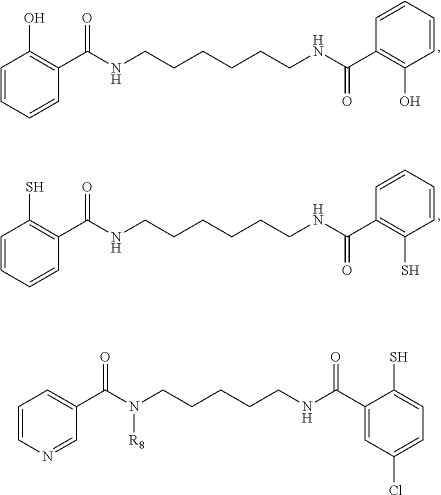

wherein R₈=H, or

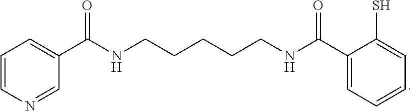 [SMILES: O=C(NCCCCCNC(=O)c1ccccc1S)c1cccnc1]

In some embodiments, the method includes the compound wherein the structure is:

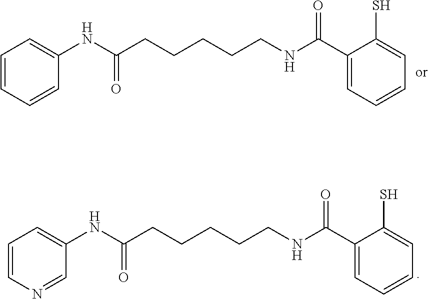 [SMILES: O=C(CCCCCNC(=O)c1ccccc1S)Nc1ccccc1 ; O=C(CCCCCNC(=O)c1ccccc1S)Nc1cccnc1]

In some embodiments, the method includes the compound having the structure

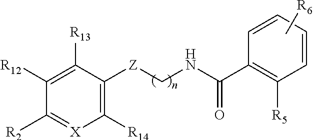

wherein
n is 3-10;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
Z is

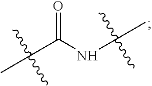 [SMILES: *NC(*)=O]

R₂ is H or NR₃R₄, wherein R₃ and R₄ are each independently H, C₄-C₆ alkyl, or C₃-C₈ cycloalkyl;
R₅ is OH or SH;
R₆ and R₁₂ are each independently H, OH, SH, F, Cl, SO₂R₁₅, NO₂, trifluoromethyl, methoxy, or CO—R₁₅, wherein R₁₅ is alkyl, alkenyl, alkynyl, C₃-C₈ cycloalkyl, or aryl; and
R₁₃ and R₁₄ are each independently H, SH, F, Cl, SO₂R₁₅, NO₂, trifluoromethyl, methoxy, or CO—R₁₅, wherein R₁₅ is alkyl, alkenyl, alkynyl, C₃-C₈ cycloalkyl, or aryl, or
a salt of the compound.

In some embodiments, the method includes the compound having the structure

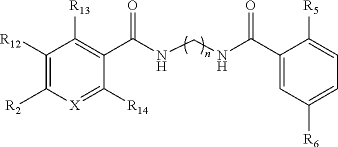

wherein
n is 3-8;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
R₂ is H or NR₃R₄, wherein R₃ and R₄ are each independently C₁-C₆ alkyl or C₃-C₈ cycloalkyl;
R₅ is OH or SH;
R₆ and R₁₂ are each independently H, OH, SH, F, Cl, SO₂R₁₅, NO₂, trifluoromethyl, methoxy, or CO—R₁₅, wherein R₁₅ is alkyl, alkenyl, alkynyl, C₃-C₈ cycloalkyl, or aryl; and
R₁₃ and R₁₄ are each independently H, SH, F, Cl, SO₂R₁₅, NO₂, trifluoromethyl, methoxy, or CO—R₁₅, wherein R₁₅ is alkyl, alkenyl, alkynyl, C₃-C₈ cycloalkyl, or aryl, or
a salt of the compound.

In some embodiments, the method includes the compound having the structure

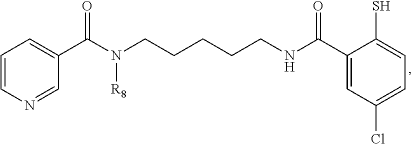

wherein R₈=H, alkyl, or aryl.

In some embodiments, the method wherein the treating comprises reducing one or more symptoms associated with traumatic brain injury in the subject.

In some embodiments, the method wherein the one or more symptoms associated with traumatic brain injury are impaired level of consciousness, impaired cognition, impaired cognitive processing speed, impaired language, impaired motor activity, impaired memory, impaired motor skills, impaired sensory skills, cerebral ischemia, edema, intracranial pressure, hearing loss, tinnitus, headaches, seizures, dizziness, nausea, vomiting, blurred vision, decreased smell or taste, reduced strength, or reduced coordination.

In some embodiments, the method wherein the treating is reducing brain tissue damage in the subject suffering from traumatic brain injury.

In some embodiments, the method wherein the treating is reducing cerebral atrophy in the subject suffering from traumatic brain injury.

In some embodiments, the method wherein the treating is increasing cerebral blood flow in the subject suffering from traumatic brain injury.

In some embodiments, the method wherein the treating is increasing cerebral glucose uptake in the subject suffering from traumatic brain injury.

In some embodiments, the method wherein the treating is reducing neuronal cell death or neuronal cell apoptosis in the subject suffering from traumatic brain injury.

In some embodiments, the method wherein the treating is reducing the loss neuronal tissue in the subject suffering from traumatic brain injury.

In some embodiments, the method wherein the treating is reducing secondary ischemia in the subject suffering from traumatic brain injury.

In some embodiments, the method wherein the traumatic brain injury is caused by a blow to the head, a penetrating injury to the head, a fall, a skull fracture, an injury due to sudden acceleration, or an injury due to sudden deceleration.

In some embodiments, the method wherein the traumatic brain injury is a penetrating head injury, a non-penetrating head injury, a skull fracture, a concussion, or a contusion.

In some embodiments, the method wherein the subject is a human.

In one embodiment, a pharmaceutical composition comprising the HDAC inhibitor. In one embodiment, a pharmaceutical composition comprising the HDAC inhibitor and a pharmaceutically acceptable carrier.

In some embodiments, a method of treating a subject suffering from traumatic brain injury comprising administering to the subject an effective amount of a pharmaceutical composition comprising the HDAC inhibitor of the present invention and a pharmaceutically acceptable carrier.

In one embodiment of the method, phosphorylation of Akt in neuronal cells in the subject is increased.

In one embodiment of the method, pro-caspase 3 expression in neuronal cells in the subject is increased.

In one embodiment of the method, after the administration of the compound, the subject's functional outcome is improved, the subject's probability of survival is increased, the progression of damage to, or ischemic damage to, or secondary ischemic damage to the brain of the subject is reduced, and/or the loss of neuronal tissue in the brain of the subject is reduced. In one embodiment, the ischemic damage is ischemic brain damage. In one embodiment, the neuronal tissue is cerebral tissue.

In some embodiments, the compound of the present invention is administered to the subject immediately following the traumatic brain injury. In some embodiments, the compound of the present invention is administered to the subject within 30 minutes, 1 hour, 6 hours, 12, 24, 48 or 72 hours following the traumatic brain injury. In some embodiments, the compound of the present invention is administered to the subject within 1 week following the traumatic brain injury.

In particular, the invention is directed to the treatment of traumatic brain injury.

As used herein, “traumatic brain injury” or “TBI” refers to any injury to the head and includes: (1) penetrating head injuries where a foreign object enters the brain and causes damage to specific brain parts, causing focal or localized damage along the route the object has traveled in the brain; and (2) closed head injuries resulting from a blow to the head, other than penetrating head injuries.

TBI causes primary brain damage, which is damage that is complete at the time of impact, including, but not limited to, skull fracture, contusions/bruises, hematomas/blood clots which may occur between the skull and the brain or inside the brain itself, lacerations duvh sd tearing of the frontal (front) and temporal (on the side) lobes or blood vessels of the brain, nerve damage (diffuse axonal injury),

TBI also causes secondary brain damage, which is damage that evolves over time after the trauma, including, but not limited to, brain swelling (edema), increased pressure inside of the skull (intracranial pressure), epilepsy, intracranial infection, fever, hematoma, low or high blood pressure, low sodium, anemia, too much or too little carbon dioxide, abnormal blood coagulation, cardiac changes, lung changes or nutritional changes.

Physical problems resulting from TBI may include, but are not limited to, hearing loss, tinnitus (ringing or buzzing in the ears), headaches, seizures, dizziness, nausea, vomiting, blurred vision, decreased smell or taste, and reduced strength and coordination in the body, arms, and legs. TBI may cause cognitive (thinking) and communication problems. TBI may cause a subject to have trouble concentrating, slower processing of new information, and/or problems with recent memory.

TBI may cause impaired level of consciousness, impaired cognition, impaired cognitive processing speed, impaired language, impaired motor activity, impaired memory, impaired motor skills, impaired sensory skills or cerebral ischemia.

In some embodiments, the traumatic brain injury comprises a mild, moderate, or severe trauma.

As used herein, a “symptom” associated with traumatic brain injury includes any clinical or laboratory manifestation associated with traumatic brain injury and is not limited to what the subject can feel or observe.

As used herein, “treatment of the diseases”, “treatment of the injury” or “treating”, e.g. of traumatic brain injury, encompasses inducing inhibition, regression, or stasis of the disease or injury, or a symptom or condition associated with the disease or injury.

As used herein, “inhibition” of disease encompasses preventing or reducing the disease progression and/or disease complication in the subject.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement, and specifically includes methyl, ethyl, propyl, butyl, pentyl, hexyl, and so on. An embodiment can be C₁-C₁₂ alkyl. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, a “compound” is a small molecule that does not include proteins, peptides or amino acids.

As used herein, an “isolated” compound is a compound isolated from a crude reaction mixture or from a natural source following an affirmative act of isolation. The act of isolation necessarily involves separating the compound from the other components of the mixture or natural source, with some impurities, unknown side products and residual amounts of the other components permitted to remain. Purification is an example of an affirmative act of isolation.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium.

The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, an “amount” or “dose” of an agent measured in milligrams refers to the milligrams of agent present in a drug product, regardless of the form of the drug product.

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to −one percent of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

### Materials and Methods

### Synthesis of 205 and 201

### Step 1: [5-(Pyridin-3-ylcarbamoyl)pentyl]carbamic acid tert-butyl ester (3)

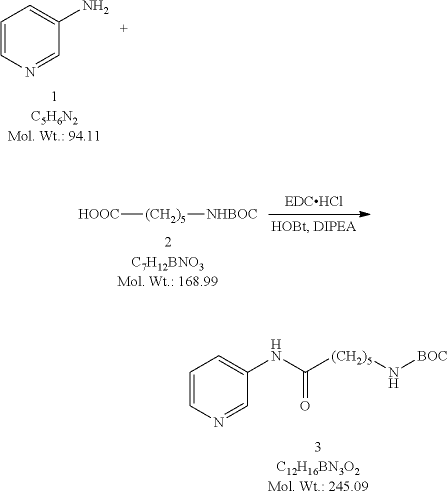

To a mixture of 3-aminopyridine (1, 2.82 g, 30 mmole) and 6-tert-Butoxycarbonylamino-hexanoic acid (2, 9.2 g, 40 mmole) in methylene chloride (50 mL) was added HOBt (135 mg, 1 mmole), EDC. HCl (7.6 g, 40 mmole) followed by DIPEA (10.45 mL, 60 mmole). The reaction mixture was stirred at room temperature for 3 h. At this point the TLC showed the disappearance of starting material. The reaction solution was washed with water (3×25 mL), followed by aqueous sodium bicarbonate (25 mL), then brine and finally dried over anhydrous sodium sulfate, filtered and concentrated. The crude residue was purified by column chromatography using 1% methanol in methylene chloride as the eluant to give the pure product as an oily residue. This residue on trituration with hexane gave 3 as a colorless solid (6.3 g, 68%, mp 96-98° C.). ¹H NMR (CDCl₃) δ 8.68 (br s, 2H), 8.48 (m, 1H), 8.30 (m, 2H), 7.32 (m, 1H), 4.62 (br s, 1H), 3.16 (m, 2H), 2.40 (m, 2H), 2.78 (m, 2H), 1.50 (m, 4H), 1.40 (s, 9H).

### Step 2: 6-Amino-N-(pyridin-3-yl)hexanoamide dihydrochloride (4)

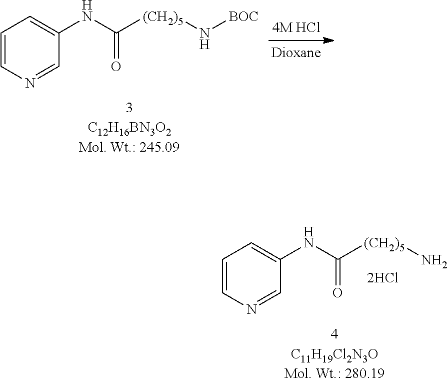

To an ice-cold mixture of [5-(Pyridin-3-ylcarbamoyl)pentyl]carbamic acid tert-butyl ester (3, 3.07 g, 10 mmole) in methylene chloride (30 mL) was added a solution of HCl in dioxane (4M, 10 mL). The mixture was stirred at room temperature overnight. The separated solid was filtered, washed with methylene chloride, dried in vacuum oven to give the product 4 as the hydrochloride salt (2.7 g, 96%). The ¹H NMR spectrum of the pure solid was consistent with structure 4. ¹H NMR (D₂O) δ 9.20 (s, 2H), 7.91 (m, 1H), 2.90 (t, 2H), 2.42 (t, 2H), 2.62 (m, 4H), 1.36 (m, 2H).

Alternative reaction conditions may be used to remove the BOC protecting group. The compound 4 can be prepared under standard amine deprotection conditions (for example, with 3.0 equivalents of 0.75M HCl (in ether), with stirring at room temperature for 12 hours (See, P. Cali, M. Begtrup, Synthesis, 2002, 63-64).

### Step 3: 2,2′-Dithio-bis{N-[5-(pyridin-3-ylcarbamoyl)pentyl]benzamide (6)

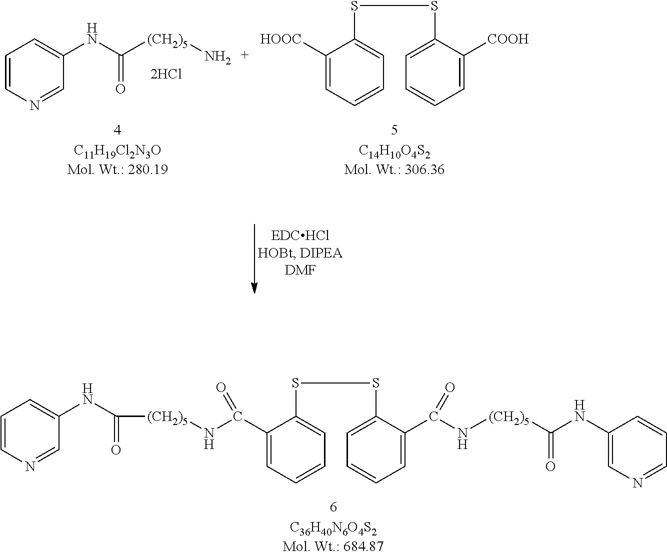

To a mixture 2-thiobenzoic acid disulfide (5, 0.765 g, 2.5 mmole), HOBt (0.665 g, 4.9 mmole), EDC. HCl (2 g, 10 mmole) in DMF (40 mL) was added the amine derivative 4 (1.5 g, 5 mmole) followed by DIPEA (3.5 mL, 20 mmole). The mixture was stirred at room temperature overnight. It was then poured into water and extracted with ethyl acetate (5×30 mL). The combined organic layers were washed with brine, dried over anhydrous sodium sulfate, filtered and concentrated. The crude residue was purified by column chromatography using 2 to 5% methanol in methylene chloride to elute the required product 6 (1 g, 27%) as a colorless solid. ¹H NMR (DMSO-d₆) δ 10.01 (br s, 2H), 8.67. (s, 2H), 8.21 (d, 2H), 7.98 (m, 4H), 7.83 (d, 2H), 7.65 (t, 2H), 7.42 (t, 2H), 7.30 (m, 2H), 3.81 (t, 4H), 2.30 (t, 4H), 1.46 (m, 4H), 1.30 (m, 4H).

The following two compounds namely 2,2′Dithio-bis{N-[5-(phenylcarbamoyl)pentyl]benzamide and 2,2′Dithio-bis{N-[5-(4-dimethylaminophenylcarbamoyl)pentyl]benzamide} [¹H NMR (CDCl₃) δ 8.00 (d, 2H), 7.56 (m, 4H), 7.35 (m, 6H), 6.66 (d, 4H), 3.90 (t, 4H), 2.90 (s, 12H), 2.51 (t, 4H), 1.76 (m, 12H), 1.45 (m, 4H)] were also synthesized using the above procedure. The ¹H NMR spectra of these two compounds are in agreement with the structures.

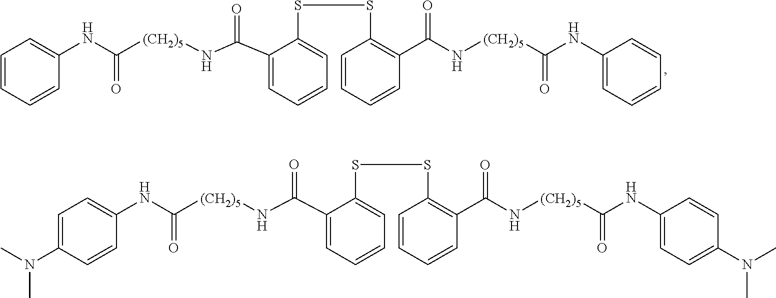

### Step 4: 2-Mercapto-N-[5-(pyridin-3-ylcarbamoyl)pentyl]benzamide (205)

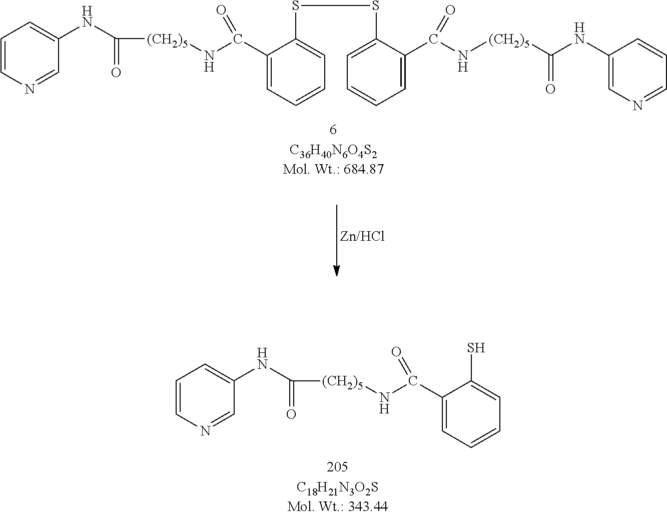

To an ice-cold solution of the disulfide derivative 6 (0.85 g, 1.2 mmole) in a mixture of methanol (10 mL) and methylene chloride (25 mL) was added conc. HCl (3.4 mL) followed by Zn dust (1.2 g) in portions over 10 minutes. After stirring at room temperature for 4 h, the mixture was diluted with water (30 mL) and methylene chloride (25 mL). The aqueous layer was separated and basified with aqueous saturated sodium bicarbonate while cooling the mixture simultaneously. The separated solid was filtered and air-dried overnight. The dried solid was extracted into a mixture of hot methanol and methylene chloride (200 mL, 2:3 ratio). The hot solution was then filtered through glass filter paper. The filtrate was evaporated to dryness and the residue was triturated with ethyl acetate to give the pure required product 205 (555 mg, 65%, mp 233-237° C.) as a colorless solid. ¹H NMR (DMSO-d₆) δ 10.06 (br s, 1H), 9.41 (br s, 1H), 8.76 (d, 1H), 8.21 (d, 1H), 8.02 (d, 1H), 7.40 (m, 2H), 7.32 (m, 1H), 7.02 (t, 1H), 6.91 (t, 1H), 3.24 (q, 2H), 2.30 (t, 2H), 1.60 (m, 4H), 1.38 (m, 2H). FAB (MH⁺) 344.

### 2-Mercapto-N-[5-(phenyl-3-ylcarbamoyl)pentyl]benzamide (201)

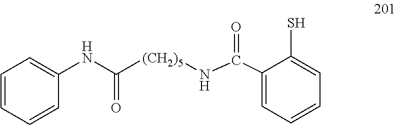

Similarly, using the above methodology and starting from aniline and 6-tert-butoxy-carbonylaminohexanoic acid (2) 2-Mercapto-N-[5-(phenyl-3-ylcarbamoyl)-pentyl]-benzamide (201) was prepared. mp 110-112° C. ¹H NMR (CDCl₃) δ 7.69 (br s, 1H), 7.58 (d, 2H, J=8 Hz), 7.49 (dd, 1H, J=6.3, 1.5 Hz), 7.35 (m, 4H), 7.16 (m, 2H), 6.41 (br s, 1H), 4.71 (s, 1H), 3.51 (q, 2H, J=6.6), 2.43 (t, 2H, J=7.2 Hz), 1.88-1.66 (m, 4H), 1.52 (m, 2H). EIMS (MH⁺) 343.

Additional HDAC inhibitors of the present invention are shown in the below table. The following compounds, including methods of preparation, are described in U.S. Pat. No. 8,143,445 B2, which is hereby incorporated by reference.

| Compound | Structure |
|---|---|
| 201 | 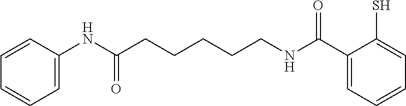 [SMILES: O=C(CCCCCNC(=O)c1ccccc1S)Nc1ccccc1] |
| 203 | 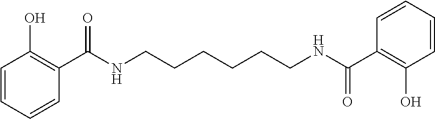 [SMILES: O=C(NCCCCCCNC(=O)c1ccccc1O)c1ccccc1O] |
| 204 | 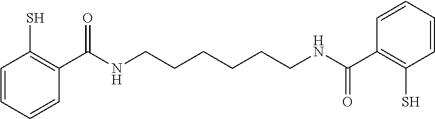 [SMILES: O=C(NCCCCCCNC(=O)c1ccccc1S)c1ccccc1S] |
| 205 | 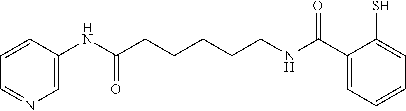 [SMILES: O=C(CCCCCNC(=O)c1ccccc1S)Nc1cccnc1] |
| 206 | 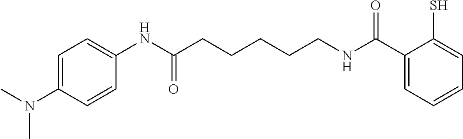 [SMILES: CN(C)c1ccc(NC(=O)CCCCCNC(=O)c2ccccc2S)cc1] |
| 207 | 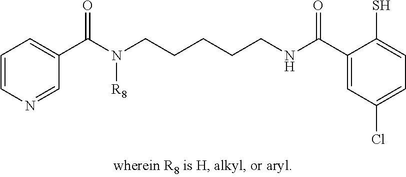 |
| 207a | 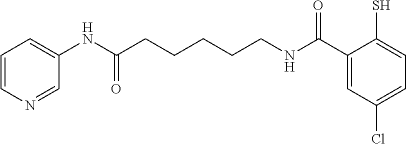 [SMILES: O=C(CCCCCNC(=O)c1cc(Cl)ccc1S)Nc1cccnc1] |
| 208 | 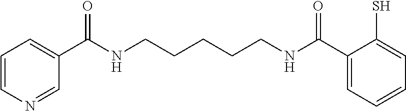 [SMILES: O=C(NCCCCCNC(=O)c1ccccc1S)c1cccnc1] |
| 209 | 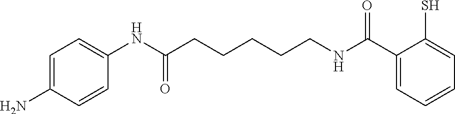 [SMILES: Nc1ccc(NC(=O)CCCCCNC(=O)c2ccccc2S)cc1] |
| 210 | 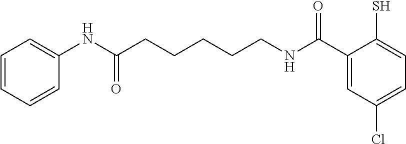 [SMILES: O=C(CCCCCNC(=O)c1cc(Cl)ccc1S)Nc1ccccc1] |
| 211 | 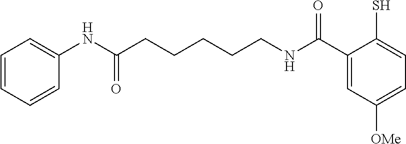 [SMILES: COc1ccc(S)c(C(=O)NCCCCCC(=O)Nc2ccccc2)c1] |
| 212 | 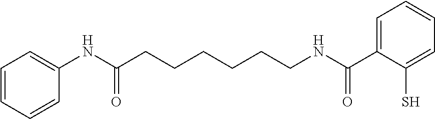 [SMILES: O=C(CCCCCCNC(=O)c1ccccc1S)Nc1ccccc1] |
| 213 | 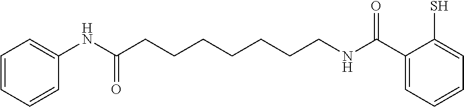 [SMILES: O=C(CCCCCCCNC(=O)c1ccccc1S)Nc1ccccc1] |
| 214 | 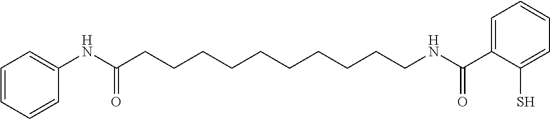 [SMILES: O=C(CCCCCCCCCCNC(=O)c1ccccc1S)Nc1ccccc1] |

### Rodent Models—Major Survival Surgery Procedure

### (1) Pre-Surgical Provisions:

Rodents are housed for 5-7 days prior to TBI or sham surgery and receive standard care. Rodents are not NPO prior to surgery.

(2) Aseptic Techniques for All Surgeries in this ASP:

The surgical instruments (for all surgeries) are sterilized with an autoclave and maintained in sterile surgical packs. The hair is removed. The skin is disinfected with 70% alcohol and chlorhexidine or povidone iodine surgical scrub. The alcohol and surgical scrub will be alternated at least three times working from the center of the proposed incision site to the periphery of the shaved area in a centrifugal pattern. The surgeon will wear a nurse cap, mask, gown and sterile gloves. Surgical supplies are autoclaved unless a heat-sensitive item needs to be sterilized with ethylene oxide gas. Between animals, the tips of the instruments are placed into a glass/ceramic bead sterilizer.

(3) Anesthesia, analgesia and Tranquilization:

Anesthesia: (Standard Isoflurane Anesthesia Plan for All Procedures) For therapy, imaging studies, and surgeries (survival or nonsurvival), rats are placed under general anesthesia in an induction chamber with 5% isoflurane delivered by oxygen. Once the animal is unconscious, and unresponsive to gentle toe pinch, anesthesia is maintained with 2-3% isoflurane (to effect) administered via nosecone or in an imaging chamber. Sterile ophthalmic ointment is applied to the corneas to prevent desiccation under anesthesia. Animals are placed on the imaging platform and Vet wrap, gauze or Kling wrap are used to secure the rat's position, hold a breathing sensor in place and prevent loss of body temperature. Care is taken to allow chest expansion for respiration. Depth of anesthesia is monitored by direct visualization of the animal and/or respiratory rate (regular, smooth respirations at the expected rate), and an adequate depth of anesthesia is confirmed by a lack of response to the pedal reflex when there is direct access to the body of the animal. The toes of the rat are pinched in an effort to elicit a spinal nerve response. A lack of response is one indicator that rat is deep enough, and a lack of motor (limb) movement is also confirmed. Body temperature is measured by rectal thermometer and maintained by heating pad. The heating pad is covered with paper towel insulation prior to laying the animal on it.

Analgesia: (Standard Post-surgical Analgesia Plan for All Procedures) Each animal receives the following to alleviate pain/distress: Acetominphen is given at a dose of 110 mg/kg orally once the rats are awake, alert and moving about their cage after surgery. If the rats hunch in the corner of their cage with their head tucked under, they are given buprenorphine at 0.01-0.05 mg/kg during their pm check. Rats may need another dose of acetominphen on day two post-surgery, but generally this is not needed.

### (4) Establish Controlled Cortical Impact (TBI):

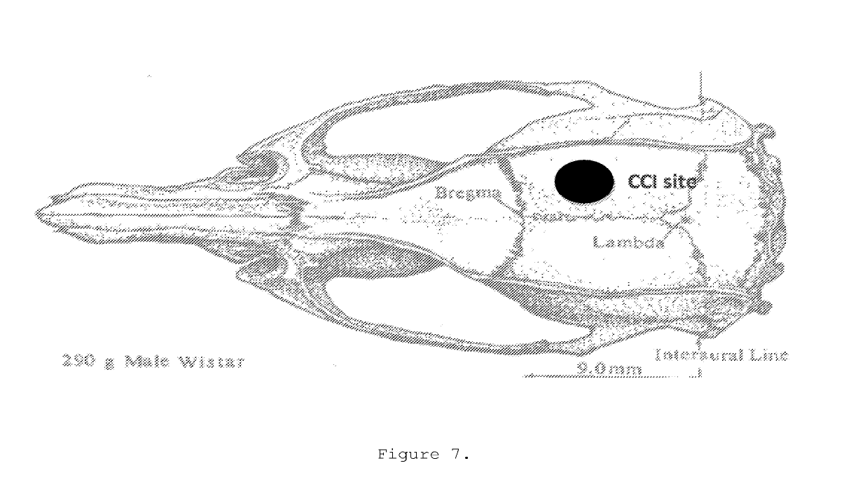

The rats are anesthetized as described above. The rodent's head is be shaved, the animal placed in a stereotaxic device, the skin is prepared as in the Aseptic Techniques described above and the skull exposed using a small surgical incision over the temporal scalp. A small surgical incision (9-10 mm), a burr hole in the skull (5-6 mm), and a single contusion over the right frontal cortex is performed (FIG. 7). A 5.0 mm burr hole is drilled into the skull with a hand-held trephine to expose the dura mater. The impact tip (5-6 mm in diameter) is slowly lowered to the dural surface where a low-voltage detector indicates when the tip contacts the dura, and contact will be visually verified. A single contusion is then made onto the surface of the dura (tip penetration depth of 2.1 mm, velocity of 5 m/s) over the left frontal cortex. Following CCI, the burr hole is covered with a bone flap, sealed with Jet Denture Repair Professional Package, and then the incision is closed with non-absorbable suture in a simple interrupted pattern. Sham-treated controls are treated similarly, but the impactor is not activated.

### (5) The Following Standard Post-Surgical Monitoring Plan is Used for all Surgeries:

Research personnel inspect all TBI rats at least twice a day for 3 days to check incision for appropriate healing and animal recovery. After 3 days, all TBI rats are monitored three times a week for clinical signs. In addition to the post-surgical monitoring of animals by research personnel, personnel monitor the animals daily during the first seven days following surgery, and if the animal is recovering uneventfully, 3 times/week until sutures are removed. Initial weight of the animal is recorded. Additional weight measurements could be recorded if needed.

### (6) Euthanasia and Disposition of Animals:

The rats are euthanized in a CO₂ chamber and death is confirmed by a secondary physical method of euthanasia such as creation of a pneumothorax, removal of a vital organ, decapitation, cervical dislocation or exsanguination. For imaging experiments, the animals are under anesthesia (isofluorane) at the end of each time point. The animals are then recovered.

### Post-Surgery Treatment and Evaluation

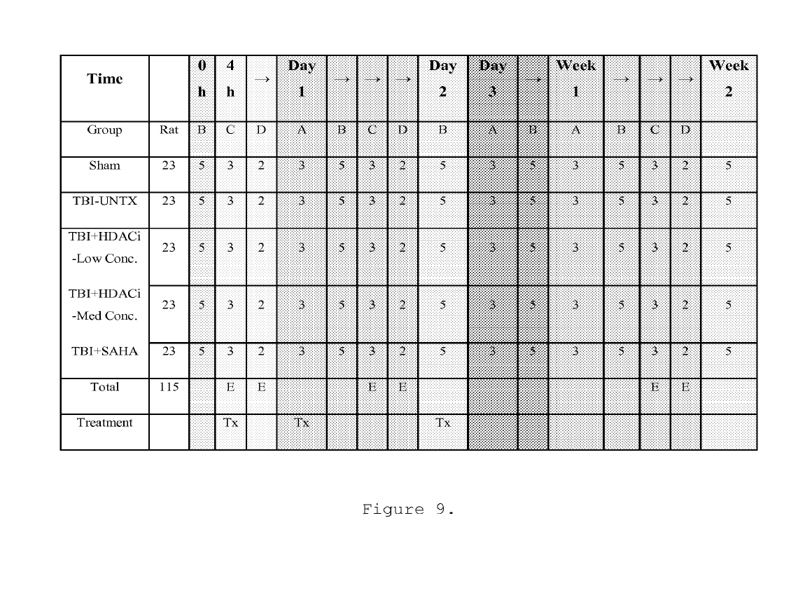

TBI rodent model animals are treated with HDACis post-surgery at 4 h, 24 h and 48 h. The efficiency of HDACi treatment is evaluated as shown in FIG. 9 by studying the following parameters:
A=FDG-PET imaging analysis
B=Rota behavior test
C=Pathology analysis (H&E, IHC, IF, & gross)
D=Frozen samples (2D gel and WB)
E=Harvest animals and procure brain samples
Tx=HDI treatment

### (1) FDG-PET Image Study:

FDG-PET imaging is conducted in larger animal cohorts treated with different drug concentrations to further confirm HDACis potential therapeutic effect in treating TBI and in increasing cerebral blood flow.

¹⁸F-FDG is purchased from the Nuclear Pharmacy of Cardinal Health, and reconstituted with sterile saline. PET scans and image analysis are performed using an Inveon microPET scanner (Siemens Medical Solutions). Each rat is injected with 18.5 MBq (500 μCi) of ¹⁸F-FDG via tail vein. All the rats are maintained under anesthesia and warmed condition. 10 min static scans are acquired at 1 h after injection. The images are reconstructed using a two-dimensional ordered-subset expectation maximum (OSEM) algorithm, and no correction is applied for attenuation or scatter. For each microPET scan, regions of interest (ROIs) are drawn over the brain and muscle region using vendor software ASI Pro 5.2.4.0 on decay-corrected whole-body coronal images. The radioactivity accumulation within brain, heart and brown fat are obtained from mean pixel values within the multiple ROI volumes and then converted to megabecquerels (MBq) per milliliter per minute using a conversion factor. These values are then divided by the administered activity to obtain (assuming a tissue density of 1 g/ml) an image-ROI-derived percent injected dose per gram (% ID/g).

### (2) Neurological Severity Score (NSS) Evaluation:

As outlined above, NSS is determined at day 1, day 7, day 14 and day 21 after CCI (or sham). Although the NSS evaluation time is determined based on previous literature, time points are adjusted if the rats are too weak to take the test. The NSS was developed to assess the clinical condition of the rodents after CCI. Points are assigned for motor functions as well as behavior. The following are assessed: ability to exit from a circle, gait on a wide surface, gait on a narrow surface, effort to remain on a narrow surface, reflexes, seeking behavior, beam walking, and beam balance. The NSS measures directly the deterioration of observable neurological status, such that a low score represents nearly intact neurological status and a high score represents severe neurological injury (Table

TABLE 1
Neurological Severity Score (NSS) evaluation
(If unable, score 1. If able, score 0.)

Points

Inability to exit from a circle (50 cm in diameter)
when left in center. Rat is considered to have exited
the circle when the forelimbs are both outside. Rat is
recovered in the center of the circle.
Within 30 minutes after injury
Within 60 minutes after injury
At >60 minutes after injury
Hemiplegia: inability of rat to resist forced changes
in position. Rat is pushed back and forth laterally by
the shoulders. It should resist equally in both
directions. Variations in resistance are fairly easy
to detect.
Loss of righting reflex. Rat is recovered lying on its
left side and should right itself.
For 30 minutes after injury
For 40 minutes after injury
For 60 minutes after injury
Flexion of hind limb when raised by the tail. Rat should
extend both hind limbs and reach upwards, hind
limbs should be straight, not flexed.
Inability to walk straight. Rat can be enticed to walk
with a hind limb pinch, food, or water.
Inability to move.
Loss of startle reflex. Rat should finch heavily in
response to a loud noise about 20 cm above the head.
Loss of pinna reflex. The external auditory meatus is
touched with a Q-tip. The rat should shake its head.
Loss of seeking behavior. A normal rat will explore
the area and sniff unknown objects. A rat with a
moderate disability or more will receive the point.
Prostration. If prostrating, score 1. If not, score
0.
Loss of placing reflexes. Rat is raised by the tail
and placed back on the ground. Each limb should reach
for the ground and should place on the floor with the
palm down. The limb should not be tucked close to the
body.
Right forelimb
Left forelimb
Right hind limb
Left hind limb
Balance beam. (1.5 cm wide) Rat is scored on how long
it can balance.
<20 sec
<40 sec
<60 sec
Beam walking. Rat can be enticed to walk with food,
water, or a hind limb pinch.
Failure on 2.5 cm wide beam.
Failure on 5.0 cm wide beam.
Failure on 8.0 cm wide beam.
Total score. Based on observed deficits, a NSS is
assessed.

### (3) Behavior Test:

Balance Beam Test: The balance beam is a test of motor coordination (3-4). It is also a useful assay for sedation and joint pathology. Several beams are available. In general the round beams are harder than the square beams, and the thinner the beam the harder the test. In this test, animals have to walk and cross a round balance beam (like across one meter bridge-bar) to test their balance and motor skills. The score is recorded compared to sham group:
100%: walk balance and cross whole balance beam.
10%-90%: walk balance and cross balance beam from 10 cm to 90 cm.
0%: walk unbalance and slip down

### (4) Gross Injury Measurements

Brain injury areas are measured and calculated post-TBI at 4 hours, day 1, and week (according to FIG. 9). Volume=Length×Width×Depth (cm).

### (5) Molecular Pathological Analysis for TBI Samples:

### DNA Microarrays

Gene expression profiling using DNA microarrays holds great promise for the future of molecular diagnostics. This technology allows, in one assay, for simultaneous assessment of the expression rate of thousands of genes in a particular sample. In cDNA microarray analysis, DNA sequences, complementary to a library of mRNA from thousands of genes, are covalently bonded to a single glass slide. The immobilized cDNA sequences serve as anchoring probes to which fluorescently tagged complimentary cDNA from the test sample hybridize (produced from extracted sample mRNA). A microarray reader displays the intensity of fluorescence at each hybridized cDNA location as a colored dot on a grid, giving a snapshot of protein expression within the cellular environment being studied.

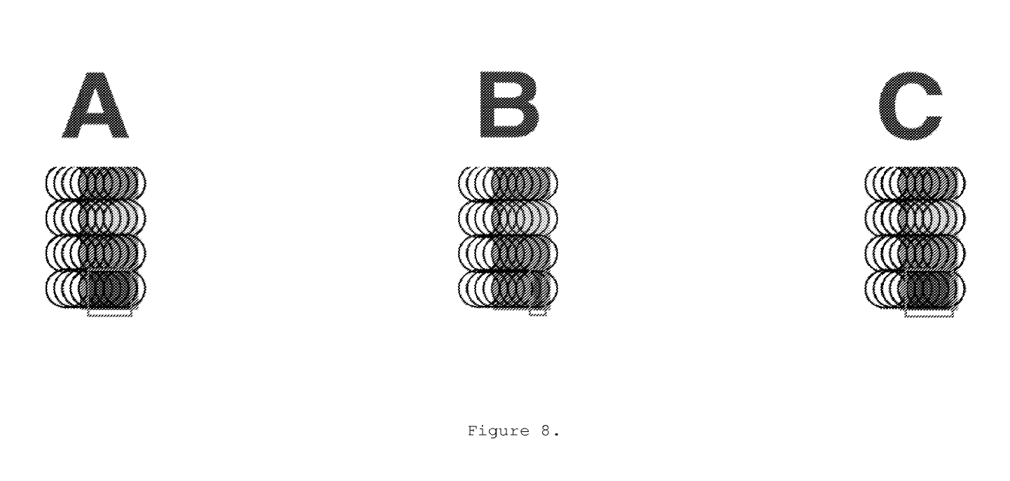

DNA microarray analysis may display different fluorescence intensities for TBI samples at different cDNA intercept locations. For example see FIG. 8: A) normal tissue, B) TBI tissue, and C) TBI+HDACi. In this hypothesis experiment, the gene expressions indicated by the red region (squared regions) would be markedly decreased in TBI tissue comparison to normal; HDACi use, however, may increase this gene expression to normal levels. Moreover, variations in gene expression help identify unique patterns that may help TBI diagnosis and predict prognosis and treatment outcome.

### Summary of DNA Microarrays Procedure

Tissue samples→Isolate total mRNA→cDNA fluorescent labeling→cDNA microarray→Hybridize→Imaging data analysis→Identify the gene changes in TBI model→Compare to normal tissue fluorescence intensities or TBI with HDACis treated tissue fluorescence intensities→Identify patterns and predict prognosis and treatment outcome

### Proteomic Analysis

Profiling of tissue specific proteins from a disease population can potentially yield valuable clinical parameters to be used for diagnosis and prognosis of the disease (Hu, S. et al. 2010). Proteomic analysis is conducted for TBI tissues to identify potential differences in protein level compared to normal tissues.

### Tissue Preparation

Rodent brain samples is collected and immediately frozen in optimal cutting temperature compound (Sakura Finetek OCT 4583). Additionally, paraffin slides are obtained for immunohistochemical study. Non-TBI normal cortex samples are also be acquired and prepared for comparison testing.

### Two-Dimensional PAGE

Tissue samples are vigorously mixed and centrifuged at 12,000 rpm to remove insoluble debris. The resulting supernatant is combined with a rehydration buffer mixture containing 8 mol/L urea, 2% CHAPS (3-[(3-cholamidopropyl) dimethylammonio]-1-propanesulfonate), 50 mmol/L dithiothreitol, and 0.2% (wt/vol) Bio-Lyte 4/7 ampholytes (163-2106, Bio-Rad); IPG (immobilized pH gradient) buffer, pH 4-7 (17-6000-86, GE Healthcare); and bromophenol blue. Rehydration is performed overnight in 11 cm pH 4-7 Immobiline Drystrips (18-1016-60, GE Healthcare) on a Reswelling Tray (GE Healthcare). Isoelectric focusing for the first dimension is performed with a Multiphore II Electrophoresis System (18-1018-06, GE Healthcare). The strips are subjected to high voltages at 300-3500 V. Immobilized pH gradient strips are equilibrated with Equilibration Buffer I containing 6 mol/L urea, 2% SDS, 375 mmol/L Tris-HCL (pH 8.8), 20% glycerol and 2% (w/v) dithiothreitol; and Buffer II containing 6 mol/L urea, 2% SDS, 375 mmol/L Tris-HCL (pH 8.8), 20% glycerol, and 2.5% (w/v) iodoacetamide (Bio-Rad, Hercules) for 15 minutes each. Precast ExcelGel SDS gels (12%-14% Gradient gel; pH 4-7, 245×180×0.5 mm; GE Healthcare) are used for the second dimension of protein separation by a Multiphor II Flated System (GE Healthcare) under a constant voltage of 700 V for 3 hours. A silver staining kit (GE Healthcare) are used according to the manufacturer's instructions to detect protein spots. All samples are run in duplicate to confirm gel electrophoretic patterning.

Intensities of protein spots on 2D gels are analyzed with Proteomweaver (Definiens) according to the manufacturer's protocol to identify if there is a statistically significant difference between TBI and normal tissues.

### Mass Spectrometry

Peptides from in-gel digests is analyzed using a ProteomeX LC/mass spectrometry system (ThermoElectron) operated in the high-throughput mode. Reversed-phase HPLC can be carried out using a BioBasic-18 column (0.18×150 mm, ThermoElectron) eluted at 1-2 μl/minute with a gradient of 2%-50% B over 30 minutes. Mobile phase A is H₂O (0.1% formic acid) and mobile phase B is CH₃CN (0.1% formic acid). Column effluent are be analyzed on the LCQ Deca XP Plus (ThermoElectron) operating in the “Top Five” mode.

### Protein Identification

Un-interpreted mass spectrometric spectra is searched against a human database using the BioWorks and SQUEST programs (ThermoElectron). Protein identification is accepted when mass spectrometric spectra of at least 2 peptides from the same protein are exhibited at a minimum default Xcorr versus charge values set by the program (for Z=1, 1.50; for Z=2, 2.00; and for Z=3, 2.50).

### Immunoprecipitation

Immunoprecipitation is performed as described previously (19). Proteins are extracted from brain tissues using IP lysis buffer with Halt proteinase inhibitor cocktail (Thermo Scientific). Total protein (400 ug) is precipitated with primary antibody (1:200) using a DynaBeads Protein G immunoprecipitation kit (Invitrogen). Proteins are precipitated overnight at 4° C. and eluted for Western blot analysis to test the samples in each group.

### Immunohistochemistry and Immunofluorescence Staining

Immunohistochemistry staining is performed using commercially available GFAP and Nestin primary antibodies (Abcam) on formalin-fixed paraffin-embedded tissue mounted on positively charged slides. The expression of Nestin is thought to identify neural stem and progenitor cells within the central nervous system. GFAP expression, however, is thought to represent astrocyte activation. Samples are labeled and visualized using a DAB staining kit (EnvisionKit; Dako, Carpinteria, Calif., USA). Immunofluorescence staining is also performed (with primary antibodies for GFAP and Nestin) in order to best assess the spatial and temporal pattern of expression of molecular markers associated with astrocyte activation and gliosis observed during the brain tissue injury repair process. The specimens are visualized using a Zeiss LSM 510 confocal microscope (Carl Zeiss, Thornwood, N.Y., USA).

### Western Blotting

Sections of frozen tissue protein from normal brain and TBI tissues in the injury area are extracted in T-PER solution, sonicated and centrifuged at 15,000 g at 4° C. to remove insoluble debris. The supernatant is used as the lysate. The protein concentration in each sample is measured by a colorimetric assay (Bio-Rad Protein Assay Kit) (Bio-Rad; Hercules, Calif.). Samples are denatured at 95° C. for 5 minutes in protein loading buffer. Equal amounts of protein at thirty micrograms of each lysate is loaded onto 4%-20% SDS-polyacrylamide gel (Invitrogen), and the proteins are electrophoretically transferred to nitrocellulose membranes and blocked with 5% milk solution. Detection of protein-bound primary antibodies is performed with a horseradish peroxidase-conjugated secondary antibody specific to rabbit or mouse immunoglobulin for one hour and an enhanced chemiluminescence system. Expression of specific proteins in each sample are determined and TBI injury tissue protein expression are compared to normal brain.

### Example 1

### FDG-PET Imaging

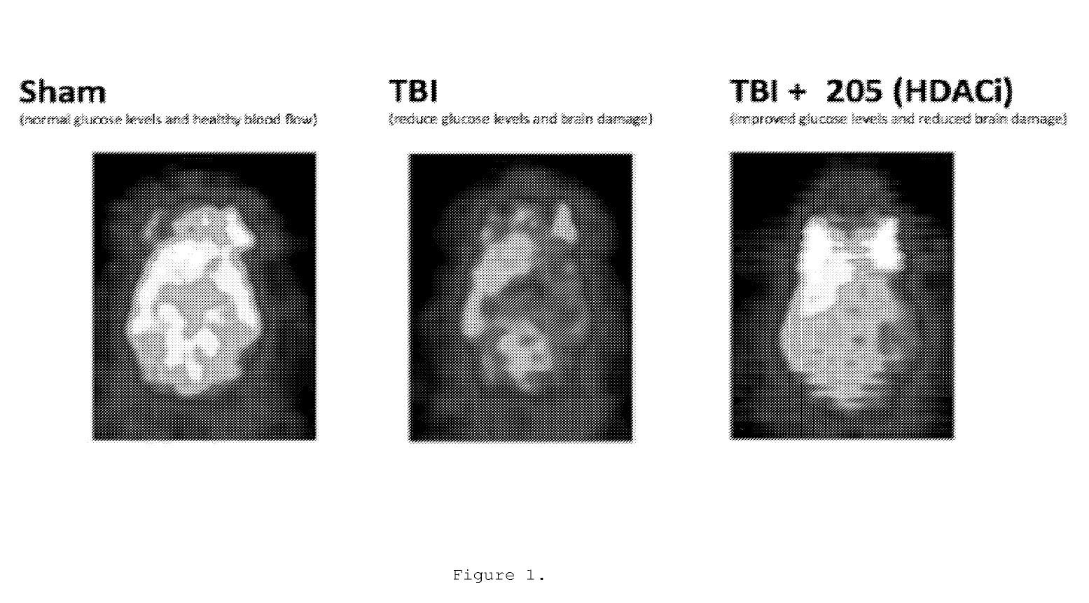

As a result of decreased cerebral blood flow, glucose uptake levels were reduced at the sight of injury in rodent brains following TBI. However, glucose levels were significantly increased in rodents treated with 205 (FIG. 1). This result indicates that HDAC inhibitors up-regulates angiogenic activity, and furthermore, also are useful in treating ischemic cerebral disease. Furthermore, FDG-PET imaging has significant potential as a non-invasive tool, it may also be useful for the diagnosis of TBI and monitoring treatment response after injury.

### Example 2

### Pathological Analysis (H & E Staining) and Gross Appearance

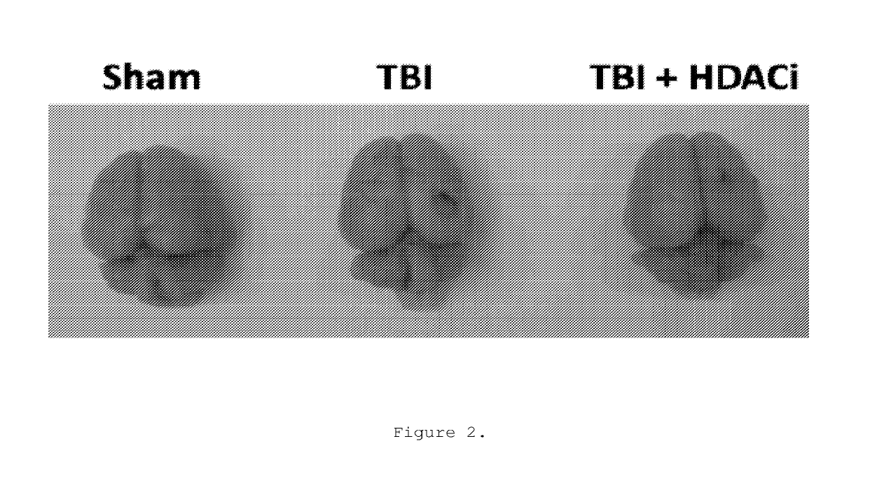

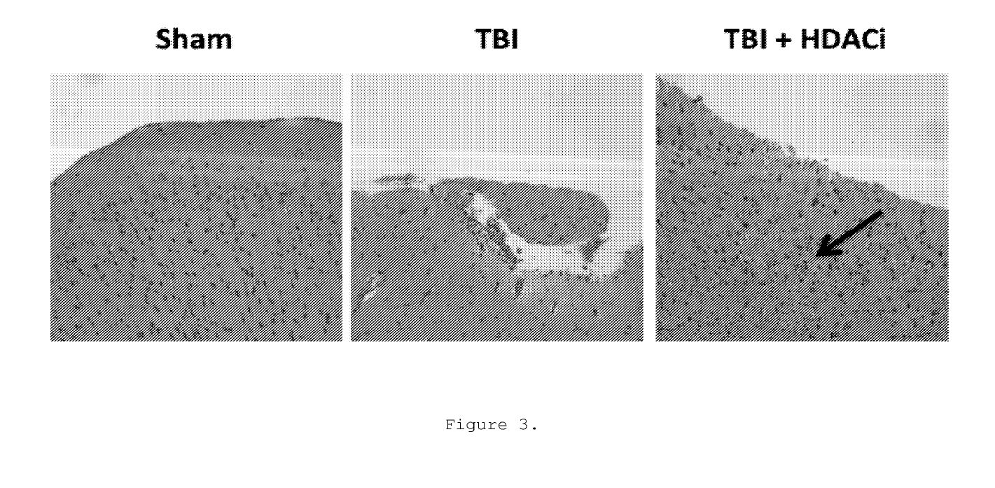

TBI induced cellular destruction and brain tissue damage (necrosis) compared to sham. 205 treated brains demonstrated much less scaring and less appearance of obvious injury during gross post-mortem examination (FIG. 2). Moreover, after 205 treatment, increased reactive gliosis (as well as increased cellularity) was observed, indicating that HDACis have a positive effect on brain tissue injury recovery and/or repair (FIG. 3).

### Example 3

### Rodent Behavior Assay

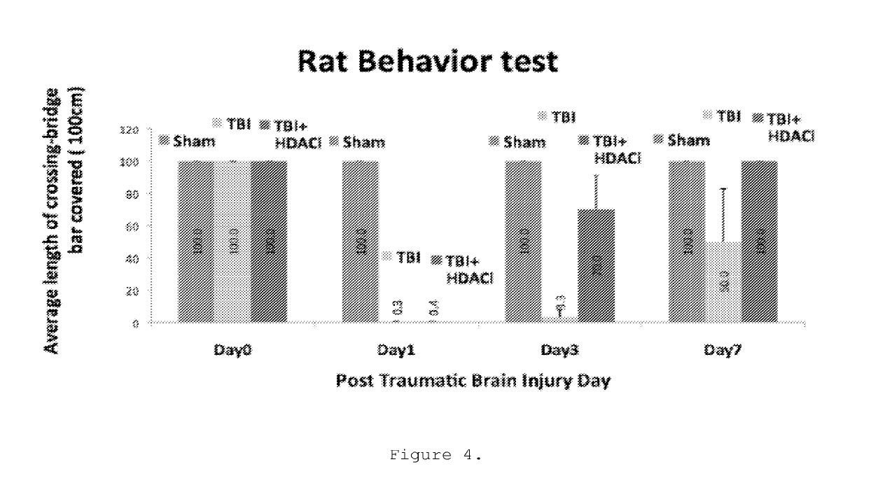

Rodents treated with HDACi (205) performed significantly better post-injury at motor skill tests (FIG. 4).

### Example 4

### Western Blot Assay

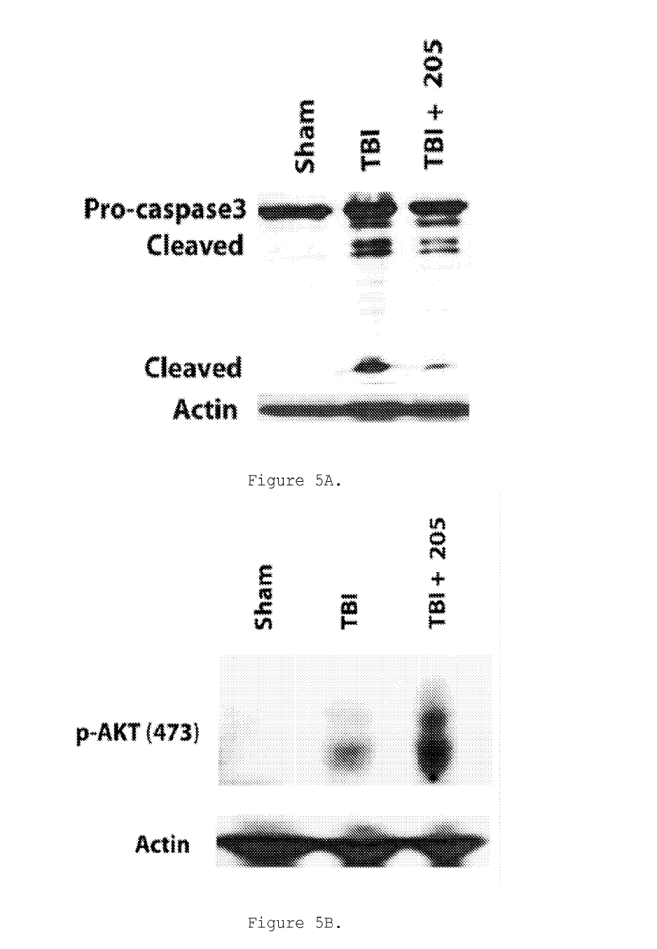

Cleaved Caspase 3 expression is significantly decreased after treatment with 205 (FIG. 5a). Therefore, 205 induces protective function in preventing neuronal cell death and apoptosis. Moreover, 205 administration increased the expression of phosphorylated-AKT (FIG. 5b), suggesting that HDACis increase neuronal cell survival.

### Example 5

### Immunofluorescence (IF) and Immunochemistry (IHC)

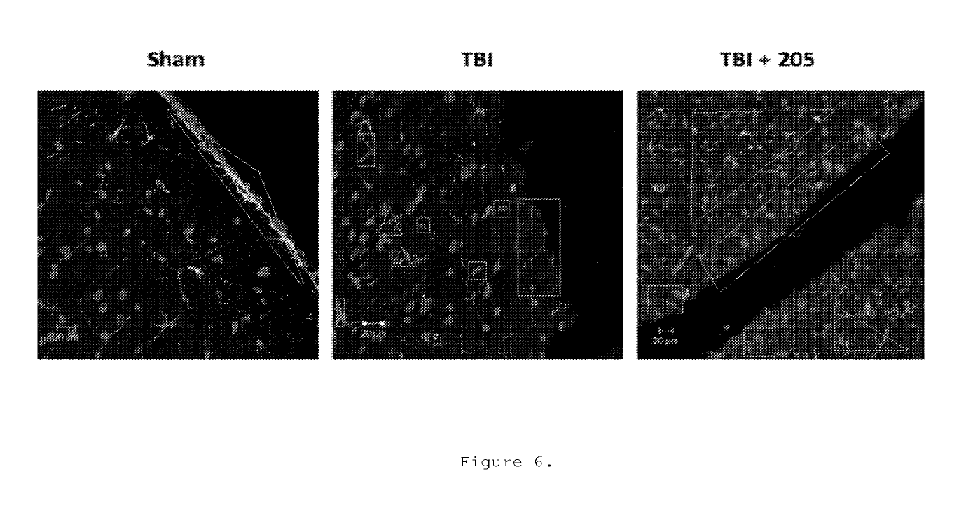

Staining for Nestin (neural stem-like cell marker) and GFAP (indicative of reactive astrocytes) was increased in those cells treated with 205, in comparison to sham and TBI untreated brains (FIG. 6). As we have shown previously, separate groups of cells (Nestin expressing proliferative neural cells with stem-like properties and GFAP expressing reactive migratory astrocytes) appear during the astrocyte injury repair response and display distinct organizational patterning (with GFAP expressing cells being most adjacent to the direct site of injury) (Yang et al. 2012). Once repair is complete, reactive astrocytes and proliferative stem-like cells revert to a quiescent state in which GFAP and Nestin expression are no longer detectable. Therefore, these results indicate that HDACis enhance the CNS molecular repair process and induce the formation of new stem-like proliferative neural cells.

### Example 6

### HDAC Inhibitors

The compounds used in the method of the present invention are HDAC inhibitors (see U.S. Pat. No. 8,143,445 B2). Compound 205 increased glucose uptake levels, decreased Pro-Caspase 3 expression and increased p-Akt expression in rat brain cells following TBI. 205 also induced formation of new stem-like proliferative neural cells following TBI. Post-TBI, rats treated with compound 205 also performed significantly better in motor skills tests. Additional HDAC inhibitors disclosed herein are expected to have activity analogous to 205.

The compounds used in the method of the present invention are HDAC inhibitors (see U.S. Pat. No. 8,143,445 B2). Compounds 201, 203, 204, 206, 2071, 207a, 208, 209, 210, 211, 212, 213 and 214 increase glucose uptake levels, decrease Pro-Caspase 3 expression and increase p-Akt expression in rat brain cells following TBI. Compounds 201, 203, 204, 206, 2071, 207a, 208, 209, 210, 211, 212, 213 and 214 also induce formation of new stem-like proliferative neural cells following TBI. Post-TBI, rats treated with compounds 201, 203, 204, 206, 2071, 207a, 208, 209, 210, 211, 212, 213 and 214 also perform significantly better in motor skills tests.

### Example 7

### Post-TBI Recovery

Compounds 201, 205, and other HDAC inhibitors disclosed herein reduce post-functional decline in human patients that have suffered a Traumatic Brain Injury. Compounds 201, 205, and other HDAC inhibitors disclosed herein induce the brain tissue injury repair response in human patients that have suffered a Traumatic Brain Injury. Compounds 201, 205, and other HDAC inhibitors disclosed herein decrease long-term neuronal dysfunction and cognitive in human patients that have suffered a Traumatic Brain Injury.

Compounds 201, 203, 204, 206, 2071, 207a, 208, 209, 210, 211, 212, 213 and 214 reduce post-functional decline in human patients that have suffered a Traumatic Brain Injury. Compounds 201, 203, 204, 206, 2071, 207a, 208, 209, 210, 211, 212, 213 and 214 induce the brain tissue injury repair response in human patients that have suffered a Traumatic Brain Injury. Compounds 201, 203, 204, 206, 2071, 207a, 208, 209, 210, 211, 212, 213 and 214 decrease long-term neuronal dysfunction and cognitive in human patients that have suffered a Traumatic Brain Injury.

### Example 8

### Symptoms of TBI

Compounds 201, 205, and other HDAC inhibitors disclosed herein reduce complications or symptoms associated with or caused by TBI. Compounds 201, 205, and other HDAC inhibitors disclosed herein reduce the effects of other dysfunctions associated with or caused by TBI including, but not limited to, impaired level of consciousness, impaired cognition, impaired cognitive processing speed, impaired language, impaired motor activity, impaired memory, impaired motor skills, impaired sensory skills or cerebral ischemia.

A subject that has suffered a traumatic brain injury is administered an compound 205 immediately after the injury has occurred. The subject is found to have a reduced loss of neuronal tissue and/or reduced ischemic brain damage as compared to a comparably injured subject who does not receive the HDAC inhibitor.

Compounds 201, 203, 204, 206, 2071, 207a, 208, 209, 210, 211, 212, 213 and 214 reduce complications or symptoms associated with or caused by TBI. Compounds 201, 205, and other HDAC inhibitors disclosed herein reduce the effects of other dysfunctions associated with or caused by TBI including, but not limited to, impaired level of consciousness, impaired cognition, impaired cognitive processing speed, impaired language, impaired motor activity, impaired memory, impaired motor skills, impaired sensory skills or cerebral ischemia.

A subject that has suffered a traumatic brain injury is administered compound 201, 203, 204, 206, 2071, 207a, 208, 209, 210, 211, 212, 213 or 214 immediately after the injury has occurred. The subject is found to have a reduced loss of neuronal tissue and/or reduced ischemic brain damage as compared to a comparably injured subject who does not receive the HDAC inhibitor.

### DISCUSSION

HDACs are known to play an essential role in the transcriptional machinery for regulating gene expression, induce histone hyperacetylation and to affect the gene expression. Therefore, it is identified here as a target of a therapeutic or prophylactic agent for diseases caused by abnormal gene expression such as inflammatory disorders, diabetes, diabetic complications, homozygous thalassemia, fibrosis, cirrhosis, acute promyelocytic leukemia (APL), organ transplant rejections, autoimmune diseases, protozoal infections, tumors, etc.

The major structural group of many HDAC inhibitors includes a hydroxamic acid component, presumed to be critical to the inhibitory activity of these molecules by their ability to bind zinc. Several other types of zinc binding groups as components of novel HDAC inhibitors are under evaluation. The HDAC inhibitors of the present invention, such as 205, utilize a mercaptobenzaminoyl group as the zinc binder in place of the hydroxamic acid. Relative to known HDAC inhibitor SAHA, LB-205 exhibits a longer half-life in vivo (Marks, P. A. 2007; Marks, P. A. 2010).

The HDAC inhibitors of the present invention are also active inhibitors of proliferation of human cancer cells. These compounds inhibit the activity of histone deacetylase 3 and histone deacetylase 4 (HDAC3 and HDAC4, respectively), and also affect the stability of N—CoR in human brain cell lines (U-87) when cells are exposed to the compounds in culture. The HDAC inhibitors of the present invention are also useful in the treatment of traumatic brain injury (TBI).

Whereas passage of the blood brain barrier is not necessarily required to impart TBI protection, some members of this group of compounds cross the blood brain barrier and inhibit HDAC activity in normal brain. Because such neural activity has beneficial effects on several models of neurodegenerative diseases, these compounds are useful as neuroprotective agents. Studies have demonstrated evidence that HDACi may also be effective neuroprotective agents, and furthermore, that there may be a role for their use in the treatment of TBI (Gaub, P. et al. 2010; Faraco, G. et al. 2006; Fischer, A. et al. 2010; Chuang, D. et al. 2009). Moreover, recent research has shown efficacy (improved cognitive and motor function) in treating traumatic brain injury (TBI) with Valproate (Class I HDACi activity) in a rat model (Dash, P. K. 2010).

TBI initiates a complex series of neurochemical and signaling changes that lead to pathological events including neuronal hyperactivity, excessive glutamate release, inflammation, increased blood-brain barrier (BBB) permeability and cerebral edema, altered gene expression, and neuronal dysfunction. The cognitive and behavioral symptoms associated with traumatic brain injury (TBI) are due to both the initial injury, and a series of progressive damages and secondary pathologies.

In a rodent model of TBI, FDG-PET imaging was used to test if post-injury administration of compound 205, an HDAC inhibitor, increased glucose levels and up-regulated angiogenic activity in the brain. Additionally, pathological analysis was used to test if post-injury administration of 205 reduced necrosis and scarring in the rodent brain. Administration of 205 increased glucose levels, up-regulated angiogenic activity and reduced scarring in the brain relative to rodents that were not treated by the HDAC inhibitor. The HDAC inhibitors of the present invention are capable of inducing injury repair (limiting scar formation) and promoting a neuroprotective effect in acute TBI, as well as decreasing long-term neuronal dysfunction and cognitive deficit, improving the quality of life in human post-TBI.

### REFERENCES

Chuang D, et al. Multiple roles of HDAC inhibition in neurodegenerative conditions. Trends in Neurosciences. 2009; 32: 591-601.
Dash P K, et al. Valproate administered after traumatic brain injury provides neuroprotection and improves cognitive function in rats. PLoS One. 2010; 5(6): 11383.
Faraco G, et al. Pharmacological inhibition of histone deacetylases by suberoylanilide hydroxamic acid specifically alters gene expression and reduces ischemic injury in the mouse brain. Mol Pharmacol. 2006; 70(6): 1876-84.
Faul M, et al. Traumatic Brain Injury in the United States: Emergency Department Visits, Hospitalizations, and Deaths. Centers for Disease Control and Prevention. National Center for Injury Prevention and Control. Atlanta, Ga., USA, 2010.
Fischer A, et al. Targeting the correct HDAC(s) to treat cognitive disorders. Trends in Pharmacological Sciences. 2010; 31: 605-617.
Gaub P, et al. HDAC inhibition promotes neuronal outgrowth and counteracts growth cone collapse through CBP/p300 and P/CAF-dependent p53 acetylation. Cell Death Differ. 2010; 17(9): 1392-408.
Gibson C L and Murphy S P. Benefits of histone deacetylase inhibitors for acute brain injury; a systematic review of animal studies. J Neurochem. 2010; 115(4): 806-813.
Hu S, Jiang J, and Wong D T. Proteomic Analysis of Saliva: 2D Gel Electrophoresis, LC-MS/MS, and Western Blotting. Methods Mol Biol. 2010; 666: 31-41.
Kumar A, Loane D J. Neuroinflammation after traumatic brain injury: Opportunities for therapeutic intervention. Brain Behav Immun. 2012 Jun. 21.
Lu J, et al. Histone deacetylase inhibitors prevent the degradation and restore the activity of glucocerebrosidase in Gaucher disease. Proc Natl Acad Sci. 2011; 108(52): 21200-5.
Lu J, et al. Decreased glucocerebrosidase activity in Gaucher disease parallels quantitative enzyme loss due to abnormal interaction with TCP1 and c-Cbl. Proc Natl Acad Sci. 2010; 107: 21665-70.
Marks P A. Discovery and development of SAHA as an anticancer agent. Oncogene. 2007; 26(9): 1351-6.
Marks P A. The clinical development of histone deacetylase inhibitors as targeted anticancer drugs. Expert Opin Investig Drugs. 2010; 19: 1049-1066.
Monneret C. Histone deacetylase inhibitors for epigenetic therapy of cancer. Anticancer Drugs. 2007; 18(4): 363-70.
Narayan R K, et al. Clinical trials in head injury. J Neurotrauma. 2002; 19(5): 503-557.
Richon V M, O'Brien J P. Histone deacetylase inhibitors: a new class of potential therapeutic agents for cancer treatment. Clin Cancer Res. 2002; 8(3): 662-4.
Yang, et al. β-Catenin signaling initiates the activation of astrocytes and its dysregulation contributes to the pathogenesis of astrocytomas. Proc Natl Acad Sci. 2012; 109(18): 6963-8.

1. A method of treating a subject suffering from traumatic brain injury comprising administering to the subject an effective amount of an HDAC inhibitor having the structure: 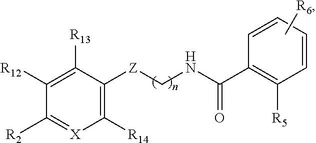
wherein
n is 1-10;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
Z is 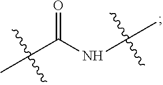 [SMILES: *NC(*)=O]
R₂ is H or NR₃R₄, wherein R₃ and R₄ are each independently H, C₁-C₆ alkyl, or C₃-C₈ cycloalkyl;
R₅ is OH or SH;
R₆, R₁₂, R₁₃, and R₁₄ are each independently H, OH, SH, F, Cl, trifluoromethyl, methoxy, or CO—R₁₅, wherein R₁₅ is alkyl, alkenyl, alkynyl, C₃-C₈ cycloalkyl, or aryl, or
a salt of the compound,
thereby treating the subject suffering from traumatic brain injury.

2. The method of claim 1, wherein the compound has the structure: 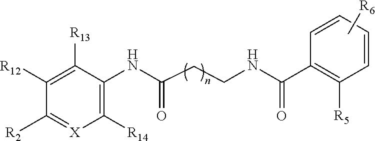
wherein
n is 1-9;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
R₂ is H or NR₃R₄, wherein R₃ and R₅ are each independently H, C₁-C₆ alkyl, or C₃-C₈ cycloalkyl;
R₅ is OH or SH; and
R₆, R₁₂, R₁₃, and R₁₄ are each independently H, OH, SH, F, Cl, SO₂R₁₅, NO₂, trifluoromethyl, methoxy, or CO—R₁₅, wherein R₁₅ is alkyl, alkenyl, alkynyl, C₃-C₈ cycloalkyl, or aryl.

3. The method of claim 2, wherein the compound has the structure: 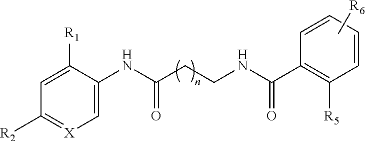
wherein
n is 1-8;
X is CH or N;
R₁ is H or OH;
R₂ is H or NR₃R₄, wherein R₃ and R₄ are each independently C₁-C₆ alkyl or C₃-C₈ cycloalkyl;
R₅ is OH or SH; and
R₆ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl.

4. The method of claim 1, wherein the compound has the structure: 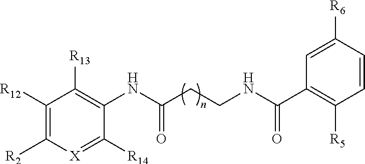
wherein
n is 1-9;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
R₂ is H or NR₃R₄, wherein R₃ and R₄ are each independently H, C₁-C₆ alkyl, or C₃-C₈ cycloalkyl;
R₅ is OH or SH; and
R₆, R₁₂, R₁₃, and R₁₄ are each independently H, OH, SH, F, Cl, trifluoromethyl, methoxy, or CO—R₁₅, wherein R₁₅ is alkyl, alkenyl, alkynyl, C₃-C₈ cycloalkyl, or aryl.

5. The method of claim 4, wherein the compound has the structure: 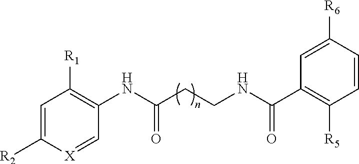
wherein
n is 1-8;
X is CH or N;
R₁ is H or OH;
R₂ is H or NR₃R₄, wherein R₃ and R₄ are each independently C₁-C₆ alkyl or C₃-C₈ cycloalkyl;
R₅ is OH or SH; and
R₆ is H, OH, SH, F, Cl, trifluoromethyl, methoxy, or CO—R₇, wherein R₇ is alkyl, alkenyl, alkynyl, or C₃-C₈ cycloalkyl, or aryl.

6. The method of claim 1, wherein the compound has the structure: 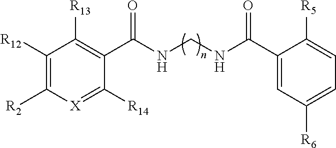
wherein
n is 1-8;
X is C—R₁₁ or N, wherein R₁₁ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl;
R₂ is H or NR₃R₄, wherein R₃ and R₄ are each independently C₁-C₆ alkyl or C₃-C₈ cycloalkyl;
R₅ is OH or SH;
R₆, R₁₂, R₁₃, and R₁₄ are each independently H, OH, SH, F, Cl, trifluoromethyl, methoxy, or CO—R₁₅, wherein R₁₅ is alkyl, alkenyl, alkynyl, C₃-C₈ cycloalkyl, or aryl.

7. The method of claim 6, wherein the compound has the structure: 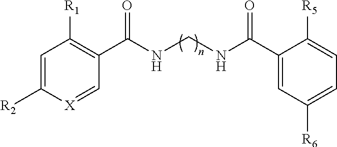
wherein
n is 3-8;
X is CH or N;
R₁ is H, OH or SH;
R₂ is H or NR₃R₄, wherein R₃ and R₄ are each independently C₁-C₆ alkyl or C₃-C₈ cycloalkyl; and
R₅ is OH or SH; and
R₆ is H, OH, SH, F, Cl, SO₂R₇, NO₂, trifluoromethyl, methoxy, or CO—R₇, wherein R₇ is alkyl, alkenyl, alkynyl, C₃-C₈ cycloalkyl, or aryl, or a salt of the compound.

8. The method of claim 1, wherein in the compound R₁ and R₂ are H, X is CH, R₅ is SH, R₆ is H, and n is 4; or
wherein in the compound R₁ is OH, R₂ is H, X is CH, R₅ is OH, R₆ is H, and n is 6; or
wherein in the compound R₁ is SH, R₂ is H, X is CH, R₅ is SH, R₆ is H, and n is 6; or
wherein in the compound R₁ and R₂ are H, X is N, R₅ is SH, R₆ is H, and n is 4; or
wherein in the compound R₁ is H, R₂ is NR₃R₄, wherein R₃ and R₄ are each C₁ alkyl, X is CH, R₅ is SH, R₆ is H, and n is 4; or
wherein in the compound R₁ and R₂ are H, X is N, R₅ is SH, R₆ is Cl, and n is 4; or
wherein in the compound R₁ and R₂ are H, X is N, R₅ is SH, R₆ is H, and n is 5; or
wherein in the compound R₁ is H, R₂ is NR₃R₄, wherein R₃ and R₄ are each H, X is CH, R₅ is SH, R₆ is H, and n is 4; or
wherein in the compound R₁ and R₂ are H, X is CH, R₅ is SH, R₆ is Cl, and n is 4; or
wherein in the compound R₁ and R₂ are H, X is CH, R₅ is SH, R₆ is methoxy, and n is 4; or
wherein in the compound R₁ and R₂ are H, X is CH, R₅ is SH, R₆ is H, and n is 5; or
wherein in the compound R₁ and R₂ are H, X is CH, R₅ is SH, R₆ is H, and n is 6; or
wherein in the compound R₁ and R₂ are H, X is CH, R₅ is SH, R₆ is H, and n is 9.

9.-20. (canceled)

21. The method of claim 1, wherein the compound has the structure: 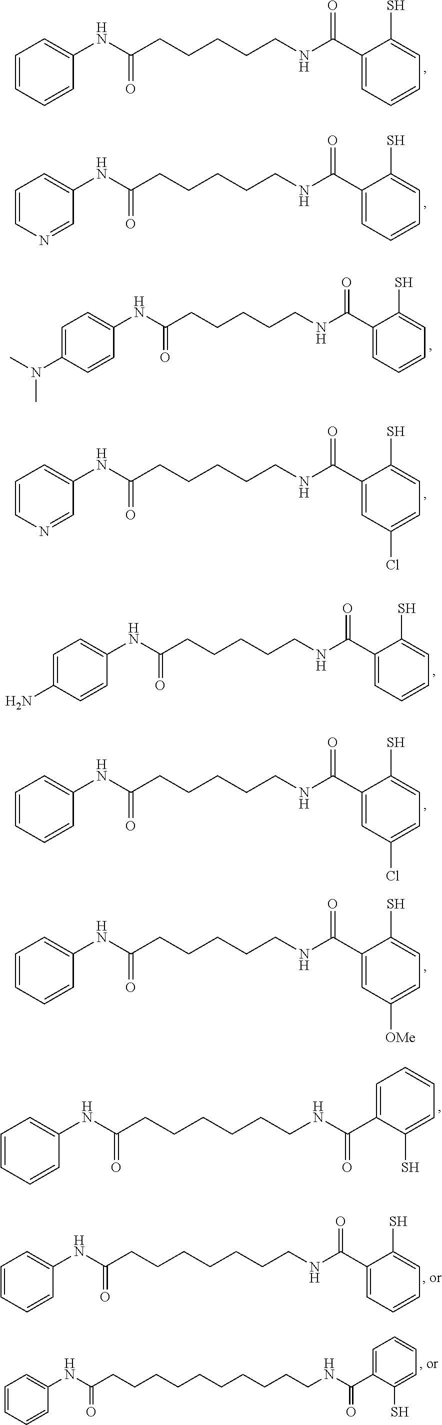
a salt of the compound.

22. The method of claim 1, wherein the compound has the structure: 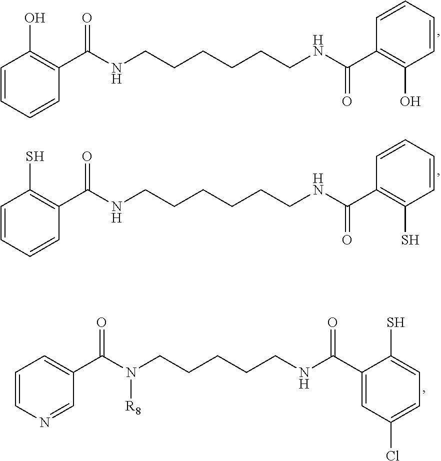
wherein R₈=H, or 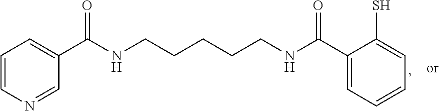 [SMILES: O=C(NCCCCCNC(=O)c1ccccc1S)c1cccnc1]
a salt of the compound.

23. The method of claim 1, wherein the compound has the structure: 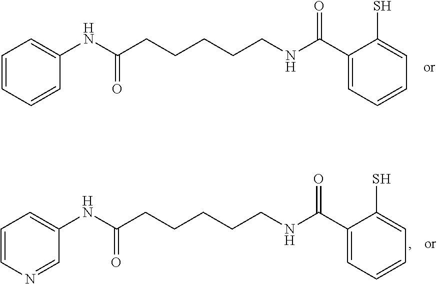 [SMILES: O=C(CCCCCNC(=O)c1ccccc1S)Nc1ccccc1 ; O=C(CCCCCNC(=O)c1ccccc1S)Nc1cccnc1]
a salt of the compound.

24. The method of claim 1, wherein the treating comprises reducing one or more symptoms associated with traumatic brain injury in the subject.

25. The method of claim 24, wherein the one or more symptoms associated with traumatic brain injury are impaired level of consciousness, impaired cognition, impaired cognitive processing speed, impaired language, impaired motor activity, impaired memory, impaired motor skills, impaired sensory skills, cerebral ischemia, edema, intracranial pressure, hearing loss, tinnitus, headaches, seizures, dizziness, nausea, vomiting, blurred vision, decreased smell or taste, reduced strength, or reduced coordination.

26. The method of claim 24, wherein the treating is reducing brain tissue damage or cerebral atrophy in the subject suffering from traumatic brain injury.

27. (canceled)

28. The method of claim 24, wherein the treating is increasing cerebral blood flow or cerebral glucose uptake in the subject suffering from traumatic brain injury.

29. (canceled)

30. The method of claim 24, wherein the treating is reducing neuronal cell death or neuronal cell apoptosis in the subject suffering from traumatic brain injury.

31. The method of claim 24, wherein the treating is reducing the loss neuronal tissue in the subject suffering from traumatic brain injury.

32. The method of claim 24, wherein the treating is reducing secondary ischemia in the subject suffering from traumatic brain injury.

33. The method of claim 1, wherein the traumatic brain injury is caused by a blow to the head, a penetrating injury to the head, a fall, a skull fracture, an injury due to sudden acceleration, or an injury due to sudden deceleration.

34. The method of claim 1, wherein the traumatic brain injury is a penetrating head injury, a non-penetrating head injury, a skull fracture, a concussion, or a contusion.

35. (canceled)
